(19) United States
(12) Patent Application Publication
(10) Pub. No.: US 2025/0256015 A1
(43) Pub. Date: Aug. 14, 2025

(54) APPARATUS AND METHOD FOR IMPROVING THE ACCURACY OF FLUID FLOW MEASUREMENT AND CONTROL

(71) Applicant: Diality Inc., Newport Beach, CA (US)

(72) Inventors: Blaine Murakami, Newport Beach, CA (US); Nicholas Hyun, Aliso Viejo, CA (US); Clayton Poppe, Irvine, CA (US); Jeff Schuster, Alameda, CA (US)

(21) Appl. No.: 18/932,374

(22) Filed: Oct. 30, 2024

Related U.S. Application Data
(60) Provisional application No. 63/546,711, filed on Oct. 31, 2023.

(51) Int. Cl. A61M 1/16; G16H 40/63

(52) U.S. Cl. CPC A61M 1/1601; G16H 40/63; A61M 2202/0021; A61M 2202/0413; A61M 2205/103; A61M 2205/3327; A61M 2205/3331; A61M 2205/3365; A61M 2205/3368; A61M 2205/50; A61M 2205/75

(57) ABSTRACT

A hemodialysis system is provided including a dialyzer, a closed loop blood flow path, a dialysate flow path, and blood and dialysate pumps. A processor controls the flow of blood through the blood flow path, and the processor controls the flow of dialysate through the dialysate flow path. The hemodialysis system includes various sensors which are connected to the processor for providing data concerning various treatment parameters. The processor monitors the various parameters of the hemodialysis machine and applies one or more prestored algorithms, algorithms created by artificial intelligence (AI) or other forms of machine learning performed by the machine or calculated remotely, to more accurately predict and control the dialysate flow rate and/or blood flow rate. Preferred parameters being monitored by the processor to improve flow rate determination and control include pump head speed, inlet and outlet pressure, tubing age (measured by pump rotations), and fluid temperature.

### CROSS-REFERENCE TO RELATED APPLICATIONS

The present application claims priority U.S. Provisional Patent Application Ser. No. 63/546,711 filed on Oct. 31, 2023, which is incorporated herein by reference.

### BACKGROUND OF THE INVENTION

The present invention relates to methods and hardware for improving fluid flow rate determinations. More particularly, the present invention is directed to determining blood flow accuracy within a hemodialysis system.

Applicant incorporates by reference herein any and all patents and published patent applications cited or referred to in this application.

Hemodialysis is a medical procedure that is used to achieve the extracorporeal removal of waste products including creatine, urea, and free water from a patient's blood involving the diffusion of solutes across a semipermeable membrane. Failure to properly remove these waste products can result in renal failure.

During hemodialysis, the patient's blood is removed by an arterial line, treated by a dialysis machine, and returned to the body by a venous line. The dialysis machine includes a dialyzer containing a large number of hollow fibers forming a semipermeable membrane through which the blood is transported. In addition, the dialysis machine utilizes a dialysate liquid, containing the proper amounts of electrolytes and other essential constituents (such as glucose), that is also pumped through the dialyzer.

Typically, dialysate is prepared by mixing water with appropriate proportions of an acid concentrate and a bicarbonate concentrate. Preferably, the acid and the bicarbonate concentrate are separated until the final mixing right before use in the dialyzer as the calcium and magnesium in the acid concentrate will precipitate out when in contact with the high bicarbonate level in the bicarbonate concentrate. The dialysate may also include appropriate levels of sodium, potassium, chloride, and glucose.

The hemodialysis process across the membrane is achieved by a combination of diffusion and convection. The diffusion entails the migration of molecules by random motion from regions of high concentration to regions of low concentration. Meanwhile, convection entails the movement of solute typically in response to a difference in hydrostatic pressure. The fibers forming the semipermeable membrane separate the blood plasma from the dialysate and provide a large surface area for diffusion to take place which allows waste, including urea, potassium, and phosphate, to permeate into the dialysate while preventing the transfer of larger molecules such as blood cells, polypeptides, and certain proteins into the dialysate.

Typically, the dialysate flows in the opposite direction to blood flow in the extracorporeal circuit. The countercurrent flow maintains the concentration gradient across the semipermeable membrane so as to increase the efficiency of the dialysis. In some instances, hemodialysis may provide for fluid removal, also referred to as ultrafiltration. Ultrafiltration is commonly accomplished by lowering the hydrostatic pressure of the dialysate compartment of a dialyzer, thus allowing water containing dissolved solutes, including electrolytes and other permeable substances, to move across the membrane from the blood plasma to the dialysate. In rarer circumstances, fluid in the dialysate flow path portion of the dialyzer has a higher pressure than the blood flow portion, causing fluid to move from the dialysis flow path to the blood flow path. This is commonly referred to as reverse ultrafiltration. Since ultrafiltration and reverse ultrafiltration can increase the risks to a patient, ultrafiltration and reverse ultrafiltration are typically conducted while supervised by highly trained medical personnel.

Unfortunately, blood flow and dialysate flow accuracy in hemodialysis machines often does not match the manufacturers' stated accuracy at high blood and dialysate flowrates. Flowrates might be lower by as much as 80-100 ml/min at a setpoint of 500 ml/min. This compares to the stated accuracy of +/−10% by most hemodialysis machine manufacturers. This difference in blood flowrate can lead to a reduction in Kt/V of 8.4%, depending on the dialysate flowrate. The hemodialysis system described herein corrects for the factors responsible for reducing the expected flowrate, such as tubing collapse due to negative inlet pressure and tubing wear, leading to more accurate control of the blood flowrate and thus a higher Kt/V that better matches the theoretical value for dose delivered. See Williams H F, Jensen K, Gillum D, Nabut J. Blood pump speed vs. actual or “compensated” blood flow rate. Nephrol Nurs J. 2007 September-October; 34(5):491-9, 525. PMID: 18041451.

A particular challenge in measuring and controlling blood flow is that parameters that are appropriate for high blood flows may not apply or be different for low flowrates.

Aspects of the present invention fulfill these needs and provide further related advantages as described in the following summary.

### SUMMARY OF THE INVENTION

According to a first aspect of the invention, a hemodialysis system is provided including an arterial blood line for connecting to a patient's artery for collecting blood from a patient, a venous blood line for connecting to a patient's vein for returning blood to a patient, a reusable dialysis machine, and a disposable dialyzer.

The arterial blood line and venous blood line may be typical constructions known to those skilled in the art. For example, the arterial blood line may be traditional flexible hollow tubing connected to a needle for collecting blood from a patient's artery. Similarly, the venous blood line may be a traditional flexible tube and needle for returning blood to a patient's vein. Various constructions and surgical procedures may be employed to gain access to a patient's blood including an intravenous catheter, an arteriovenous fistula, or a synthetic graft.

Preferably, the hemodialysis system includes three primary pumps which include two “dialysate” pumps connected to the dialysate flow path for pumping dialysate through the dialysate flow path, and a blood pump connected to the blood flow path. Preferably, a first dialysate pump is positioned in the dialysate flow path “upflow”, (meaning prior in the flow path) from the dialyzer while the second dialysate pump is positioned in dialysate flow path “downflow” (meaning subsequent in the flow path) from the dialyzer. The blood pump pumps blood from a patient through the arterial blood line, through the dialyzer, and through the venous blood line for return to a patient. It is preferred that the blood pump be positioned upflow from the dialyzer.

Preferably, the hemodialysis system contains sensors for monitoring hemodialysis. Preferably, the sensors include a motor sensor connected to the dialysate pumps and/or the blood pump. The motor sensors monitor the rotational velocity of the motors, and their corresponding rotors. Furthermore, the motor sensors track the number of revolutions that have been completed by the rotors during a hemodialysis treatment. With a pretreatment understanding as to how motor velocity and the number of cycles impacts the fluid flow through the dialysate and/or blood tubing, one can monitor any aging effects as a result of wear, and cyclic compression and decompression of the tubing so as to improve measurement accuracy and control of the dialysate and blood flows.

In addition, it is preferred that the hemodialysis machine contains one or more pressure sensors for detecting the pressure within the dialysate flow path, or at least an occlusion sensor for detecting whether the dialysate flow path is blocked. Preferably, the dialysis machine also possesses one or more pressure sensors for measuring the pressure within the blood flow path. As explained below, preferably, the hemodialysis system includes at least a pressure sensor (Pba) in the blood flow path upstream of the blood pump. The pressure sensors may incorporate flow rate sensors so that pressure and flow rate measurements may be made by a single sensor.

The hemodialysis system possesses a processor containing the electronics dedicated for controlling the hemodialysis system. The processor contains power management and control electrical circuitry connected to the pump motors, valves, and dialysis machine sensors for controlling proper operation of the hemodialysis system. To this end, the hemodialysis system's processor includes memory which stores one or more prestored algorithms, algorithms created in real time such as by artificial intelligence (AI) or other forms of machine learning performed by the machine or calculated remotely, to more accurately predict and control the dialysate flow rate and/or blood flow rate. The one or more algorithms incorporate various hemodialysis machine parameters which are monitored by the various sensors, and the processor applies the algorithms to control the dialysate and/or blood pumps' head speed. Preferred parameters being monitored by the processor to improve dialysate and/or blood flow rate determination and control include pump head speed, inlet and outlet pump pressure, tubing age (measured by pump rotations), and fluid temperature.

Advantageously, the hemodialysis system provides more accurate fluid flow through the dialysate flow path and/or blood flow path.

In addition, the hemodialysis system provides an extraordinary amount of control and monitoring not previously provided by hemodialysis systems so as to provide enhanced patient safety.

Moreover, the hemodialysis system produces a higher Kt/V that better matches the theoretical value for dose delivered.

Other features and advantages of the present invention will be appreciated by those skilled in the art upon reading the detailed description, which follows with reference to the Drawings.

### BRIEF DESCRIPTION OF THE DRAWINGS

FIG. 1 is a front view of a peristaltic pump within a hemodialysis machine;

FIG. 2 is a graph illustrating stroke volume decreases with an increase in vacuum pressure at the blood pump inlet (Pba);

FIG. 3 is a graph illustrating a α (the ratio of actual stroke volume to the stroke volume at zero pressure) as a function of upstream pressure P;

FIG. 4 is a graph illustrating that speed alters pump volume at different pressures;

FIG. 5 is a first graph illustrating that increased pump speeds result in greater error which is affected at different pressures;

FIG. 6 is a second graph illustrating that increased pump speeds result in greater error which is affected at different pressures;

FIG. 7 is a is a third graph illustrating that increased pump speeds result in greater error which is affected at different pressures;

FIG. 8 is a fourth graph illustrating that increased pump speeds result in greater error which is affected at different pressures;

FIG. 9 is a graph illustrating that speed alters pump volume at different pressures;

FIG. 10 is a graph illustrating that the scatter in the error data increases as the motor rotation rate increases;

FIG. 11 is a graph illustrating the a (the ratio of actual stroke volume to the stroke volume at zero pressure) has a distinct linear trend when pressure is considered;

FIG. 12 is a graph illustrating two test runs of tube aging (as measured by rotor rotations) showing error increasing with rotor rotations;

FIG. 13 is the graph shown in FIG. 12 with a third test run of tube aging (as measured by rotor rotations) showing the consistency of the model;

FIG. 14 is a graph illustrating a Matlab model based on the average Y intercept and the evolving slope of a vs. P as a function of rotor rotations with the result at time T=0;

FIG. 15 is a graph illustrating the Matlab model based on the average Y intercept and the evolving slope of a vs. P as a function of rotor rotations with the result at time T=2.5 hours;

FIG. 16 is a graph illustrating the Matlab model based on the average Y intercept and the evolving slope of a vs. P as a function of rotor rotations with the result at time T=5.0 hours;

FIG. 17 is a graph illustrating implementation of an algorithm to correct blood flow through a hemodialysis system at a low (100 ml/min) blood flow rate; and

FIG. 18 is a graph illustrating implementation of an algorithm to correct blood flow through a hemodialysis system at a high (500 ml/min) blood flow rate.

### DETAILED DESCRIPTION OF THE INVENTION

The invention includes apparatus and methods for more accurately determining the rate of fluid flow through medical tubing. More particularly, the invention includes methods and apparatus for more accurately determining the rate of dialysate flow and blood flow through medical tubing passing through a hemodialysis machine's peristaltic pumps. Furthermore, the invention includes using the increased accuracy of fluid flow measurements to maintain, increase, or decrease a pump's velocity to ensure that flow rate is at, or within, desired flow parameters.

A typical hemodialysis system includes an arterial blood line for connecting to a patient's artery for collecting blood from a patient, a venous blood line for connecting to a patient's vein for returning blood to a patient, a reusable dialysis machine and a disposable dialyzer. The arterial blood line and venous blood line may be typical constructions known to those skilled in the art. For example, the arterial blood line may be traditional flexible hollow tubing connected to a needle for collecting blood from a patient's artery. Similarly, the venous blood line may be a traditional flexible tube and needle for returning blood to a patient's vein. Various constructions and surgical procedures may be employed to gain access to a patient's blood including an intravenous catheter, an arteriovenous fistula, or a synthetic graft.

Preferably, the disposable dialyzer has a construction and design known to those skilled in the art including a blood flow path and a dialysate flow path. The term “flow path” is intended to refer to one or more fluid conduits, also referred to as passageways, for transporting fluids. The conduits may be constructed in any manner as can be determined by those skilled in the art, such as including flexible medical tubing or non-flexible hollow metal or plastic housings. The blood flow path transports blood in a closed loop system by connecting to the arterial blood line and venous blood line for transporting blood from a patient to the dialyzer and back to the patient. Meanwhile, the dialysate flow path may include a single pass system where a large supply of dialysate feeds dialysate at a predetermined flow rate to the dialyzer, and then the used dialysate is discarded. Alternatively, the dialysate flow path transports dialysate in a closed loop system from a supply of dialysate to the dialyzer and back to the dialysate supply. Both the blood flow path and the dialysate flow path pass through the dialyzer, but the flow paths are separated by the dialyzer's semipermeable membrane.

Preferably, the hemodialysis system contains a reservoir or chamber, such as a balancing chamber, for storing a dialysate solution. The reservoir connects to the hemodialysis system's dialysate flow path to form a closed loop system for transporting dialysate from the reservoir to the hemodialysis system's dialyzer and back to the reservoir. Alternatively, the hemodialysis system possesses two (or more) dialysate reservoirs which can be alternatively placed within the dialysate flow path. When one reservoir possesses contaminated dialysate, dialysis treatment can continue using the other reservoir while the reservoir with contaminated dialysate is emptied and refilled. The reservoirs may be of any size as required by clinicians to perform an appropriate hemodialysis treatment. However, it is preferred that the two reservoirs be the same size and sufficiently small so as to enable the dialysis machine to be easily portable. Acceptable reservoirs are 0.5 liters to 6.0 liters in size. Where the hemodialysis system includes only one reservoir, an acceptable reservoir has a volume of 12.0 liters. Alternatively, the hemodialysis system may include no reservoirs and instead use a balancing chamber to control fluid in an out of the dialyzer in a single-pass configuration.

The hemodialysis system preferably possesses one or more heaters thermally coupled to the reservoirs for heating dialysate stored within the reservoir. In addition, the hemodialysis system includes temperature sensors for measuring the temperature of the dialysate within the system. The hemodialysis system preferably possesses a fluid level sensor for detecting the level of fluid in the reservoir. The fluid level sensor may be any type of sensor for determining the amount of fluid within the reservoir. Acceptable level sensors include magnetic or mechanical float type sensors, conductive sensors, ultrasonic sensors, optical interfaces, and weight measuring sensors such as a scale or load cell for measuring the weight of the dialysate in the reservoir.

Preferably, the hemodialysis system includes three primary pumps. The first and second “dialysate” pumps are connected to the dialysate flow path for pumping dialysate through the dialysate flow path from a reservoir to the dialyzer and back to the reservoir. Preferably, a first pump is positioned in the dialysate flow path “upflow”, (meaning prior in the flow path) from the dialyzer while the second pumps is positioned in dialysate flow path “downflow” (meaning subsequent in the flow path) from the dialyzer. Meanwhile, the hemodialysis system's third primary pump is connected to the blood flow path. This “blood” pump pumps blood from a patient through the arterial blood line, through the dialyzer, and through the venous blood line for return to a patient. It is preferred that the third pump be positioned in the blood flow path, upflow from the dialyzer. Alternatively, the system may configure the dialysate pumps such that one provides fluid flow to a balancing chamber and the other disrupts fluid from that balanced system to provide ultrafiltration control.

The hemodialysis system may also contain one or more sorbent filters for removing toxins which have permeated from the blood plasma through the semipermeable membrane into the dialysate. Filter materials for use within the filter are well known to those skilled in the art. For example, suitable materials include resin beds including zirconium-based resins. Acceptable materials are also described in U.S. Pat. No. 8,647,506 and U.S. Patent Publication No. 2014/0001112.

In a first embodiment, the sorbent filter is connected to the dialysate flow path downflow from the dialyzer so as to remove toxins in the dialysate prior to the dialysate being transported back to a reservoir. In a second embodiment, the filter is outside of the closed loop dialysate flow path, but instead is positioned within a separate closed loop “filter” flow path that selectively connects to either one of the two dialysate reservoirs. For this embodiment, preferably the hemodialysis system includes an additional fluid pump for pumping contaminated dialysate through the filter flow path and its filter.

Preferably, the hemodialysis system includes two additional flow paths in the form of a “drain” flow path and a “fresh dialysate” flow path. The drain flow path includes one or more fluid drain lines for draining the reservoirs of contaminated dialysate, and the fresh dialysate flow path includes one or more fluid fill lines for transporting fresh dialysate from a supply of fresh dialysate to the reservoirs. One or more fluid pumps may be connected to the drain flow path and/or a fresh dialysate flow path to transport the fluids to their intended destination.

In addition, the hemodialysis system includes a plurality of fluid valve assemblies for controlling the flow of blood through the blood flow path, for controlling the flow of dialysate through the dialysate flow path, and for controlling the flow of used dialysate through the filter flow path. The valve assemblies may be of any type of electro-mechanical fluid valve construction as can be determined by one skilled in the art including, but not limited to, traditional electro-mechanical two-way fluid valves and three-way fluid valves. A two-way valve is any type of valve with two ports, including an inlet port and an outlet port, wherein the valve simply permits or obstructs the flow of fluid through a fluid pathway. Conversely, a three-way valve possesses three ports and functions to shut off fluid flow in one fluid pathway while opening fluid flow in another pathway. In addition, the hemodialysis system's valve assemblies may include safety pinch valves, such as a pinch valve connected to the venous blood line for selectively permitting or obstructing the flow of blood through the venous blood line. The pinch valve is provided so as to pinch the venous blood line and thereby prevent the flow of blood back to the patient in the event that an unsafe condition has been detected.

Preferably, the hemodialysis system contains sensors for monitoring hemodialysis. To this end, preferably the hemodialysis system has at least one flow sensor connected to the dialysate flow path for detecting fluid flow (volumetric and/or velocity) within the dialysate flow path. In addition, it is preferred that the dialysis machine contain one or more pressure sensors for detecting the pressure within the dialysate flow path, or at least an occlusion sensor for detecting whether the dialysate flow path is blocked. Preferably, the dialysis machine also possesses one or more sensors for measuring the pressure and/or fluid flow within the blood flow path. The pressure and flow rate sensors may be separate components, or pressure and flow rate measurements may be made by a single sensor.

Furthermore, it is preferred that the hemodialysis system includes a blood leak detector (“BLD”) which monitors the flow of dialysate through the dialysate flow path and detects whether blood has inappropriately diffused through the dialyzer's semipermeable membrane into the dialysate flow path. In a preferred embodiment, the hemodialysis system includes a blood leak sensor assembly incorporating a light source which emits light through the dialysate flow path and a light sensor which receives the light that has been emitted through the dialysate flow path. After passing through the dialysate flow path, the received light is then analyzed to determine if the light has been altered to reflect possible blood in the dialysate. In addition, the dialysis machine may possess a bubble sensor connected to the arterial blood line and a bubble sensor connected to the venous blood line for detecting whether gaseous bubbles have formed in the blood flow path.

The hemodialysis system preferably includes additional sensors including an ammonia sensor and a pH sensor for detecting the level of ammonia and pH within the dialysate. Preferably, the ammonia sensor and pH sensor are in the dialysate flow path immediately downstream of the filter.

The hemodialysis system possesses a processor containing the dedicated electronics for controlling the hemodialysis system. The processor contains power management and control electrical circuitry connected to the pump motors, valves, and dialysis machine sensors for controlling proper operation of the hemodialysis system. To this end, the hemodialysis system's processor includes software and memory which are pre-programmed to store one or more patient treatment plans by which a patient is treated.

Furthermore, the processor memory includes data and algorithms for more accurately determining and maintaining fluid flow rates through the hemodialysis system in accordance with the patient treatment plans. More particularly, the processor includes data and algorithms for more accurately determining and controlling the dialysate and blood flow rates through the hemodialysis system. In turn, the processor monitors various parameters of the hemodialysis machine and applies the prestored algorithms, or algorithms created by artificial intelligence (AI) or other forms of machine learning, to more accurately predict the dialysate flow rate and/or blood flow rate. The AI created algorithms may be created within the hemodialysis machine. Preferred parameters being monitored by the processor include:
Fluid Flow Velocity (Pump Head Speed)
Inlet and Outlet Pressures
Tubing Aging
Fluid temperature

By monitoring these operating parameters and applying corrective algorithms, the hemodialysis system's processor adjusts (to maintain, increase or decrease) the pump head's speed to correct for the factors responsible for reducing the expected flowrate, such as tubing collapse due to negative inlet pressure and tubing wear. By adjusting for the operational parameters, the hemodialysis system provides more accurate control of the blood flowrate and thus a higher Kt/V that better matches the theoretical value for doses delivered.

The basic equation for flow rate is: Flow Rate=Stroke Volume*Stroke Speed. However, as explained in greater detail below, various operational parameters introduce error into this calculation, and unfortunately hemodialysis machines, and medical pumps in general, typically calculate flow rate only by measuring pump stroke speed in conjunction initial stroke volume.

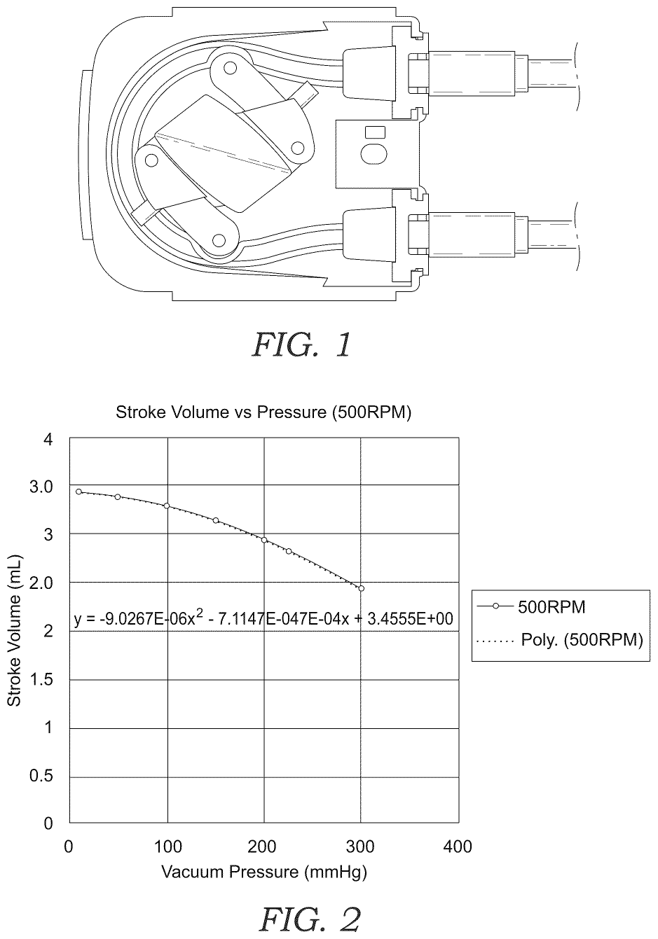

To correct for these errors, numerous hemodialysis machine operational parameters were examined using a hemodialysis machine including peristaltic pumps, as illustrated in FIG. 1, and employing Tygon medical tubing part no. ND-100-65 ADF00017 (clear) having an 0.375″ outer diameter (OD) and an 0.250″ inner diameter (ID). For purposes herein, “pressure” refers to the transmural pressure, i.e., the pressure inside the tubing relative to the ambient pressure. The “tubing inside the pump” is used throughout this document rather than the simpler “pump tubing” as “pump tubing”. A preferred hemodialysis machine employed peristaltic pumps including two rollers resulting in twice the stroke volume per rotation, and a gear reduction of 32 from the motor to the rotor.

### Inlet and Outlet Pressures)

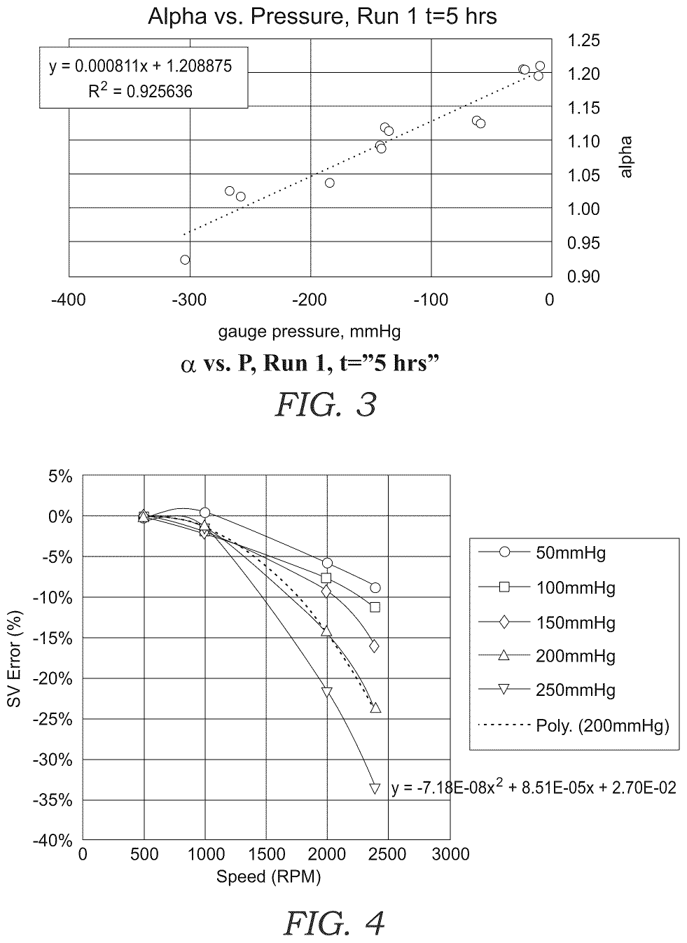

For purposes herein, “PBA” and “Pba” are acronyms for (pressure, blood, arterial) which refer to pressure measured by the pre-blood pump pressure sensor. As illustrated in FIG. 2, stroke volume decreases with an increase in vacuum pressure at the Pba, with testing occurring in the axis-symmetric deformation region for this 25 inHg (635 mmHg) tube (pre-buckling). This results in a corresponding error illustrated in FIG. 3 wherein a is the ratio of actual stroke volume to the stroke volume at zero pressure as a function of upstream pressure P. As explained in greater detail below, this is a low-speed data that does not consider rebound recovery effects as a function of speed/pressure (therefore additional pressure compensation is assessed related to speed dependence).

As illustrated in FIGS. 2 and 3, the inlet fluid pressure significantly affects flow rate and measurement accuracy. However, it has been found that stroke volume is not affected by downstream pressure (backpressure) for the clinically applicable operating range (<1% change in flow as a function of change in pressure over pressure range from 0-600 mm Hg). Accordingly, downstream pressure may, but does not necessarily need to, be considered in correcting fluid flow calculations. Furthermore, as explained in greater detail below, the inlet pressure affects flow rate more at higher speeds than at lower speeds.

### Blood Flow Velocity (Pump Head Speed)

Pump speed has been found to have a significant impact on the accuracy of fluid flow determination. Specifically, pump speed can affect stroke volume because of the following: rebound recovery time; pressure; temperature; axial strain variation (pull effect); and thermal variation impact on tube elasticity (impacts deformation as a function of pressure). As illustrated in graphs FIG. 4, speed alters pump volume at different pressures. This compensating for speed (2nd order poly) on the 200 mmHg curve (this is the 60601-2-16 test pressure and a relevant clinical (max) pressure) provides improved fluid flow determinations. Further, this data shows that one must only compensate for speeds greater than 1500 RPM (as for <1500 RPM error is <5%).

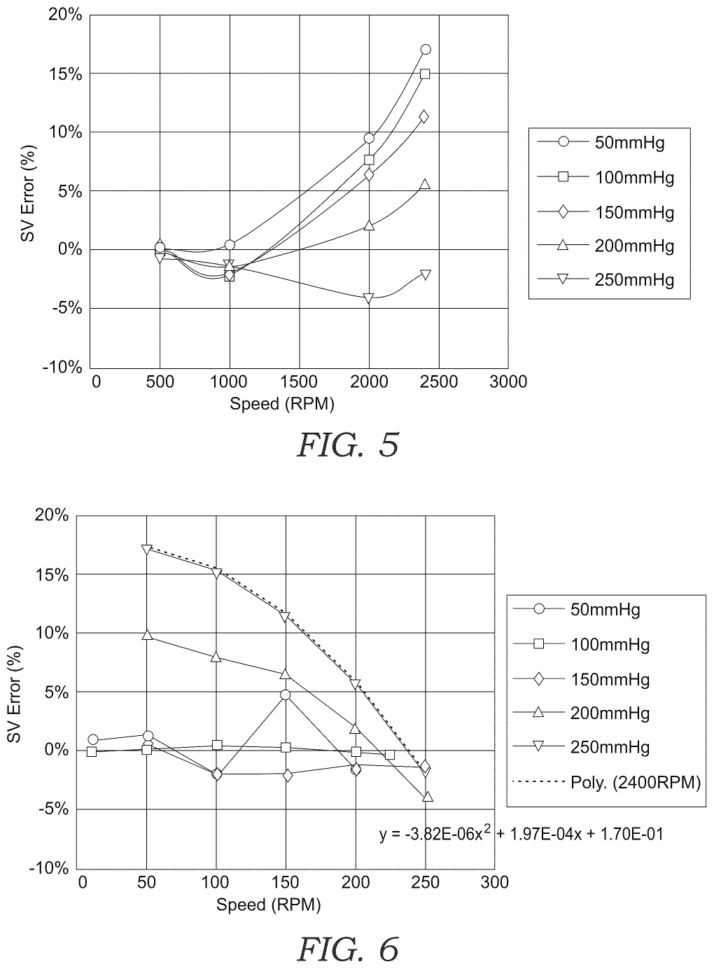

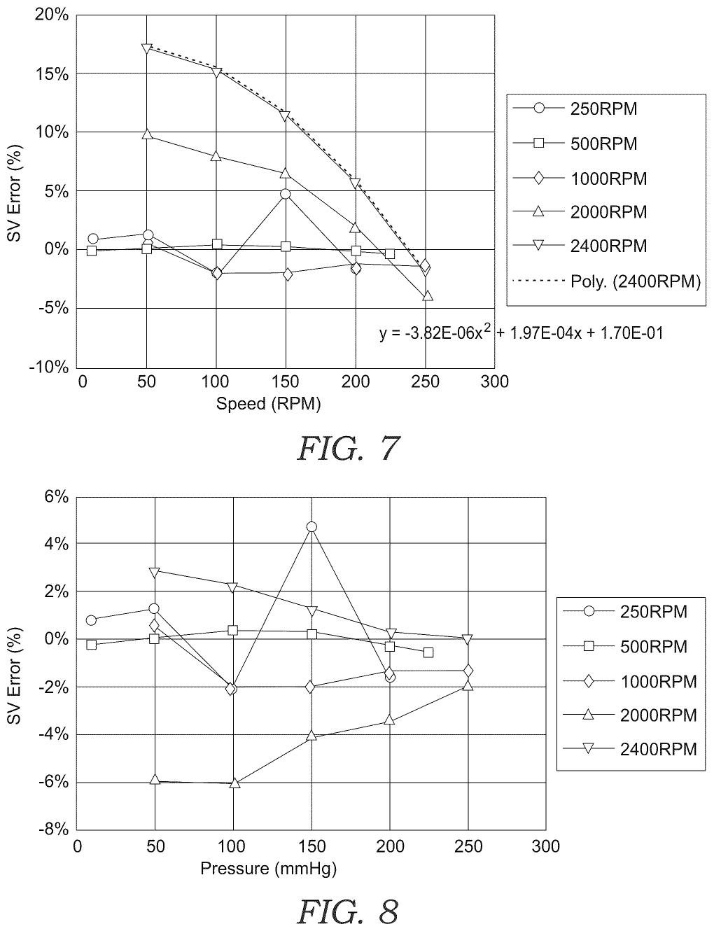

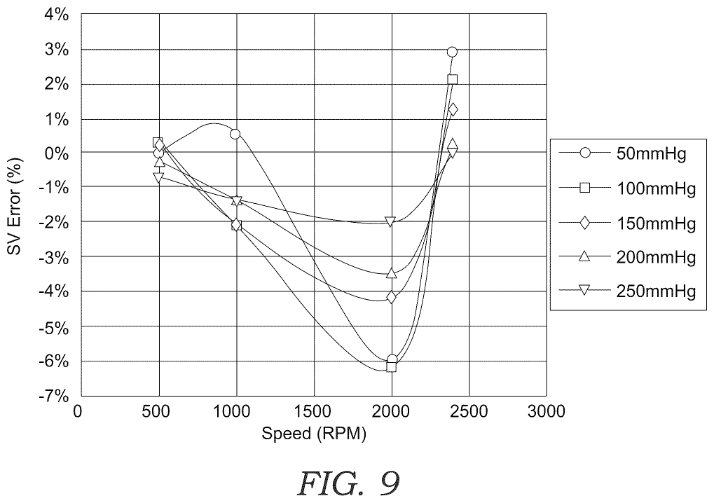

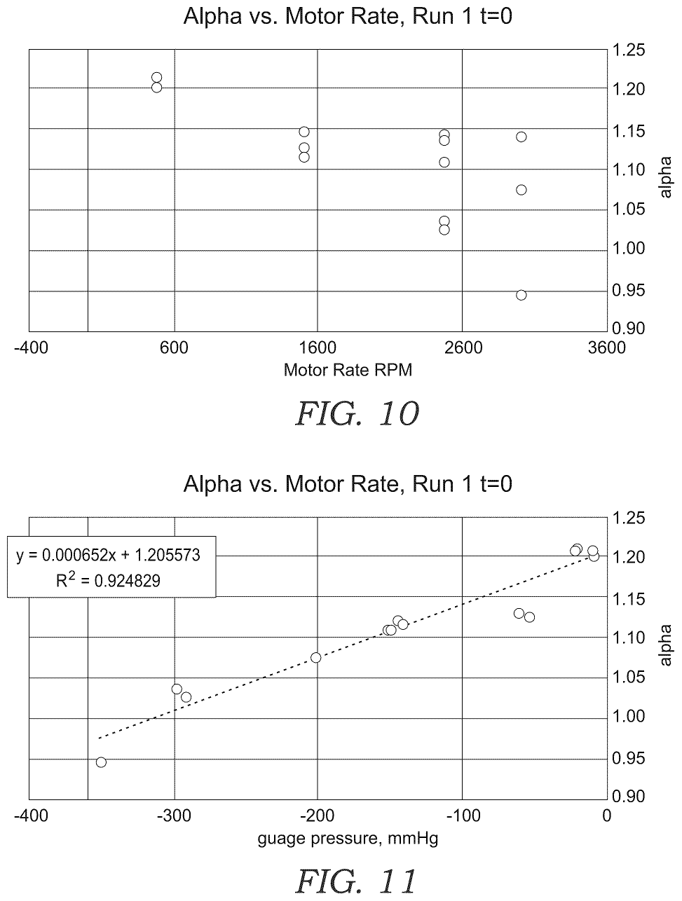

Furthermore, FIGS. 5-8 show that increased pump speeds result in greater error which is further affected at different pressures. As illustrated in FIGS. 9 and 10, the scatter in the data increases significantly as the motor rotation rate increases. Thus, it is difficult to determine a clean linear trend by considering motor rotation rate alone. However, as illustrated in FIG. 11, the a (the ratio of actual stroke volume to the stroke volume at zero pressure) has a distinct linear trend when pressure is considered.

Accordingly, preferred algorithms for predicting blood flow velocity include consideration of both pump speed and arterial pressure. The foregoing shows that need to transition from used to not used compensation in a way that is not abrupt (weighted average on a transition range). For this embodiment, and with reference to FIGS. 9-11, the following may be considered for estimating fluid flow:
PCSV—Pressure corrected stroke volume
PC2—Pressure compensation coefficient (−0.0000090267)
PC1—Pressure compensation coefficient (−0.00071147)
NSV—Nominal stroke volume (3.4555)
SCSV—Speed corrected stroke volume
SC2—Speed compensation coefficient (−0.0000000718)
SC1—Speed compensation coefficient (0.0000851)
SC0—Speed compensation coefficient (−0.027)
PSCSV—Additional pressure compensated stroke volume (higher pressures)
PSC2—Pressure compensation coefficient (−0.00000382)
PSC1—Pressure compensation coefficient (0.000197)
PSC0—Pressure compensation coefficient (0.17)
TS0—Start of weighted speed transition (1000)
TS1—End of weighted speed transition (1500)
P—Vacuum Pressure (mmHg) (Filtered—filtering is TBD)
S—Motor Speed (RPM) (set speed, assume set speed=actual speed)

This produces the following equations.

$$\cdot PCSV = PC 2 * P \hat{} 2 + PC 1 * P + NSV$$

$$\cdot SCSV = PCSV ( 1 + SC 2 * S \hat{} 2 + SC 1 * S + SC 0 )$$

$$\cdot PSCSV = SCSV ( 1 + PSC 2 * P \hat{} 2 + PSC 1 * P + PSC 0 )$$

LSWF—Low Speed Weighting Factor
HSWF—Low Speed Weighting Factor
PSCSVT—Pressure Corrected Speed Corrected Stroke Volume with weighted transition full range

$$\cdot HSWF = [ if [ ( S - TS 0 ) > 0 , ( S - TS 0 ) , 0 ] - [ if [ ( S - TS 1 ) > 0 , ( S - TS 1 ) , 0 ] ] / [ TS 1 - TS 0 ]$$

$$\cdot LSWF = 1 - HSWF$$

$$\cdot PSCSVT = LSWF * PCSV + HSWF * PSCSV$$

### Tubing Aging (Pump Rotations)

In addition to pump speed and inlet pressure, the age (meaning cyclic compression and decompression) of the medical tubing within the peristaltic pump affects the accuracy of fluid flow calculations. The medical tubing used for the dialysate and blood flow paths undergoes cyclic fatigue from the peristaltic pumps that alters the elasticity of the tubing. This “aging” affects the accuracy of dialysate and blood flow rate measurements. In addition, it has been found that the impact of tube aging is also a function of input pressure and its effect on accurately estimating fluid flow.

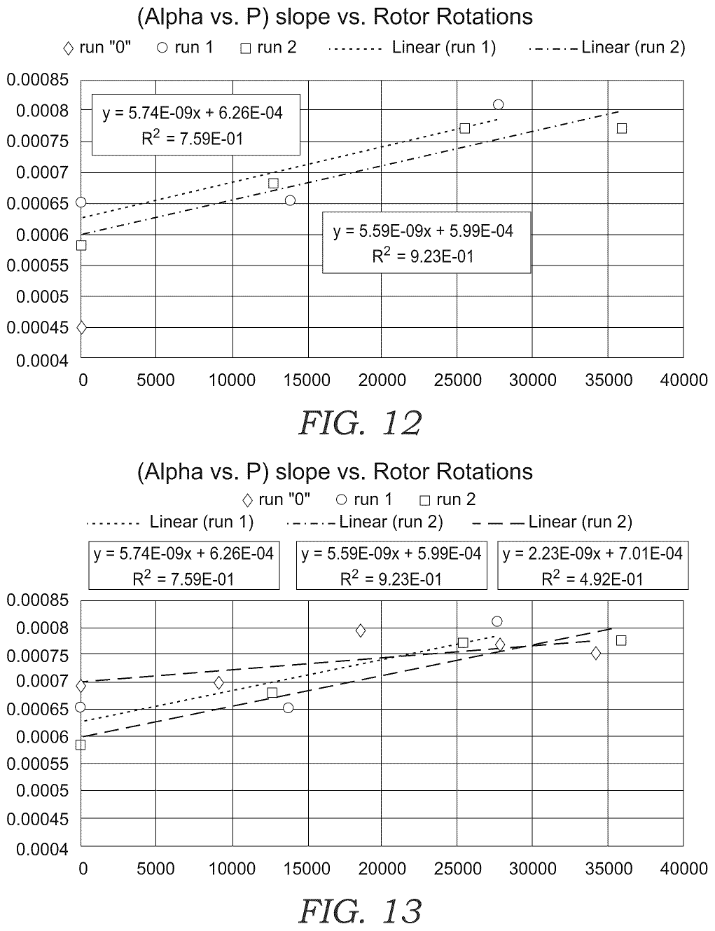

As illustrated in FIGS. 12 and 13, multiple tests of tube aging (as measured by rotor rotations) show error increasing with rotor rotations. Advantageously, the multiple tests produced very similar slopes. Accordingly, algorithms considering rotor rotations can be employed to improve predicted fluid flow.

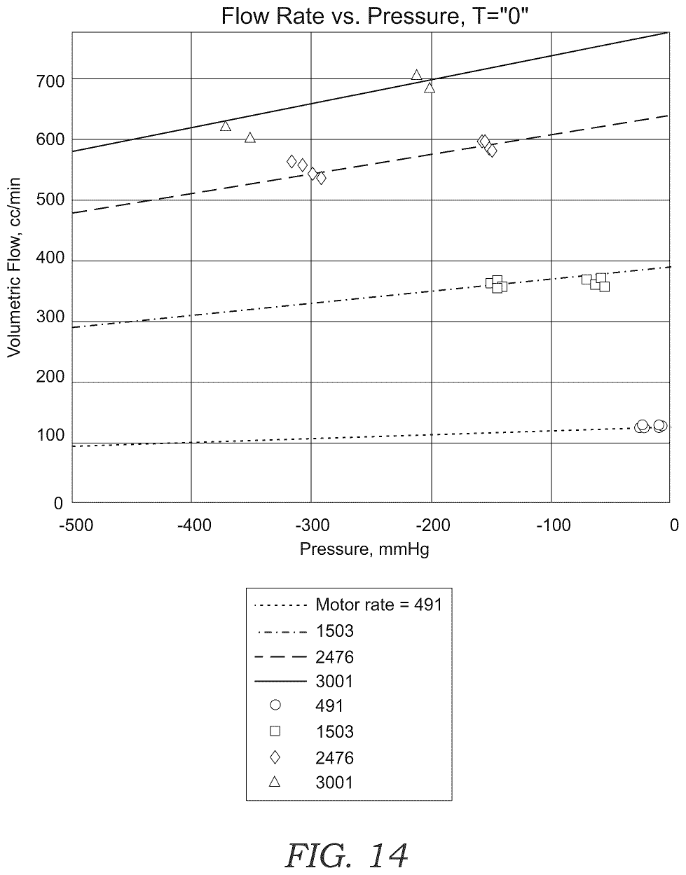

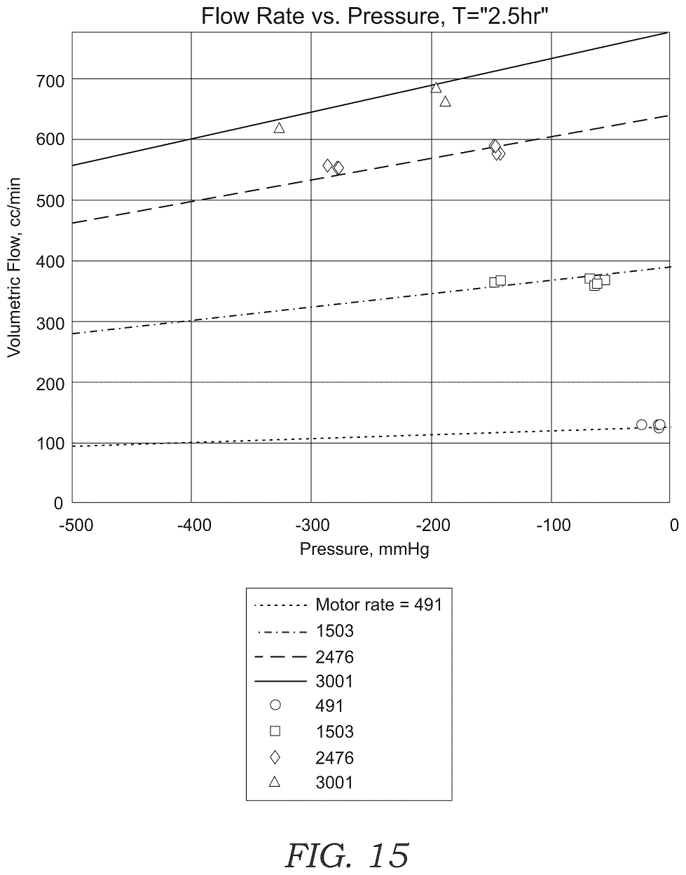

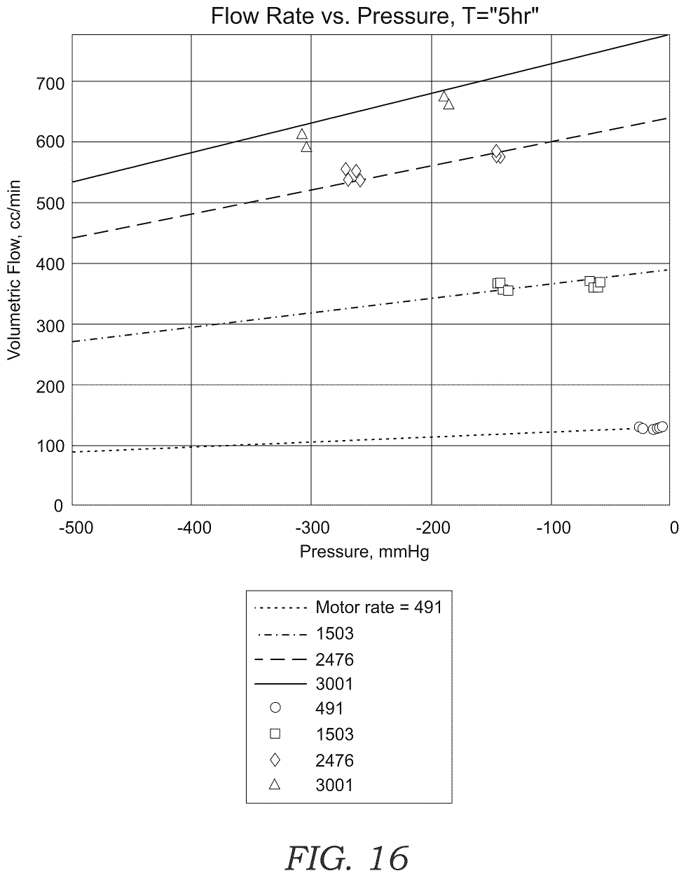

Moreover, measuring inlet pressure in combination with tube aging can produce even more accurate algorithms for predicting fluid flow. To this end, a Matlab model was developed based on the average Y intercept and the evolving slope of a vs. P as a function of rotor rotations with the results at the various time points, as shown in FIGS. 14-16. The data are included for comparison to the model. It can be seen that the model does a reasonably good job of predicting the data, and thus the model is a candidate for correcting the flow rate based on aging (rotor rotations) in combination with the measured pressure.

### Model Implementation

A preferred algorithm for use with increasing the accuracy of fluid flow determinations included consideration of the following.

| Parameter | New Tubing |
|---|---|
| 2*SV (Q calculated) | 6.81 |
| aₒ | 1.197 |
| m′ | 2.90E−08 |
| mₒ | 5.50E−04 |

This produces the formula:

QB=Motor RPM*6.81((2.90E−08*motor rotations+5.50E−04)*Pba pressure+1.197

An alternative algorithm is explained as follows. It has been determined that an estimate of the flow rate \dot{Q} can be calculated as a function of the rotor rotation rate ωᵣₒₜₒᵣ, the pump input pressure P, and the number of rotor rotations n since the tubing was inserted. However, what is desired for controlling the pump is the inverse function, i.e., the rotor rotation rate ωᵣₒₜₒᵣ as a function of the number of rotor rotations n and input pressure P that will yield the desired flow rate \dot{Q}.

In the model above, we have the following relationship:

$$2 * SV = 6 \text{.81} \qquad Equation 1$$

Where SV is the stroke volume

$$\dot{Q} = 2 * SV * {ω}_{rotor} * α ( n , P ) \qquad Equation 2$$

Where α is a linear function of the pump input pressure P:

$$α ( n , P ) = m ( n ) * P + {α}_{0} \qquad Equation 3$$

And the slope m(n) is a linear function of the number of motor rotations n:

$$m ( n ) = {m}^{′} * n + {m}_{0} \qquad Equation 4$$

Combining these expressions yields:

$$\dot{Q} = 2 * SV * {ω}_{rotor} * ( ( {m}^{′} * n + {m}_{0} ) * P + {α}_{0} ) \qquad Equation 5$$

Thus, the desired equation is:

$${ω}_{rotor} = \frac{\dot{Q}}{2 * SV * ( ( {m}^{′} * n + {m}_{0} ) * P + {α}_{0} )} \qquad Equation 6$$

2*SV=6.81 (see Equation 1). α₀ can be taken as the average Y intercept of α for run 1, at t=0, or (1.2056 (see FIG. 11). Similarly, m′=(5.74+5.59)/(2*10⁹), and m₀=(6.26+5.99)/(2*10⁴) (see FIG. 12).
Substituting the above into Equation 6 above yields:

$${ω}_{rotor} = \frac{\dot{Q}}{6.81 * ( 5.665 * 1 {0}^{- 9} * n + 6.13 * 1 {0}^{- 4} ) * P + 1.2056}$$

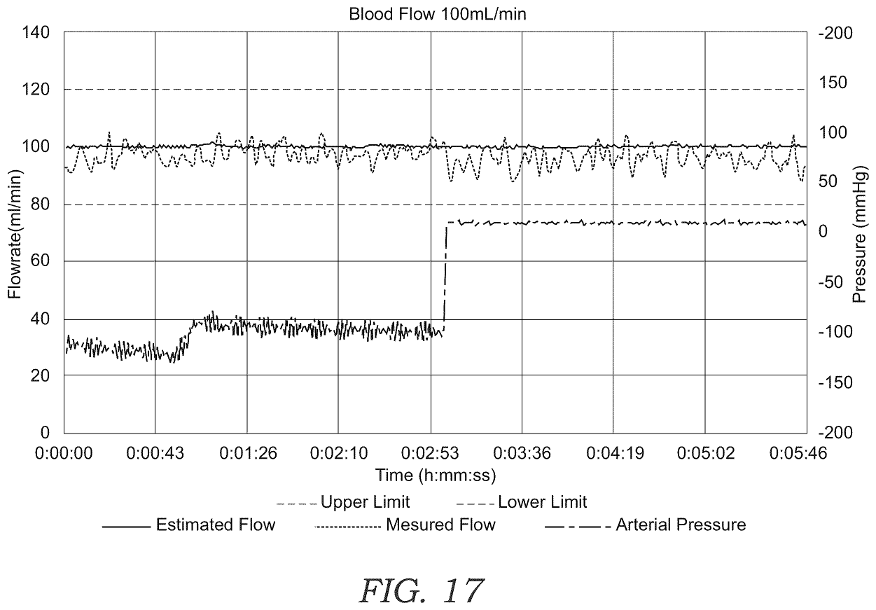

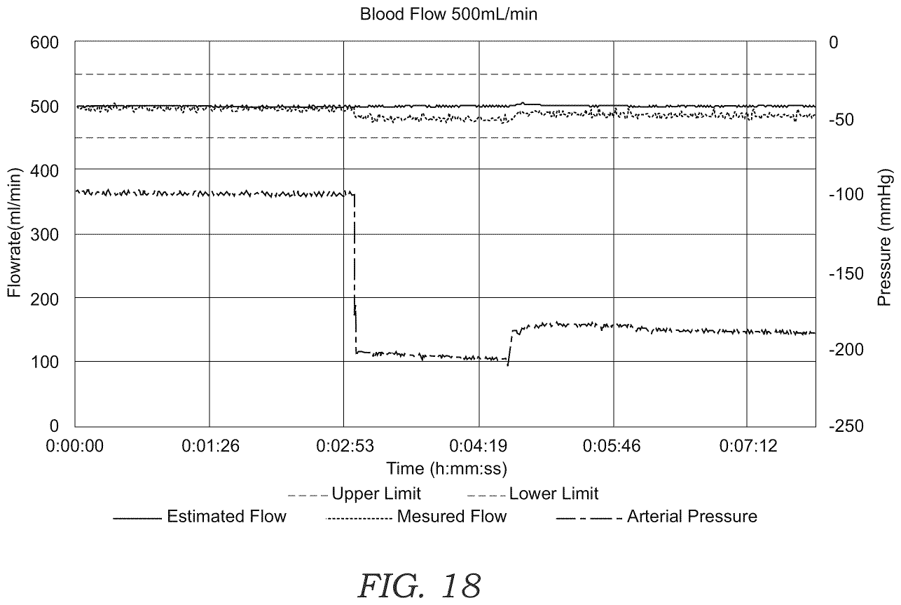

With reference to FIGS. 17 and 18, even with significant changes in arterial pressure, which should affect flow rate accuracy, implementation of the algorithm accurately controls flow so as to be within +/−10% of intended flow for both low (100 ml/min) and high (500 ml/min) blood flow rates. Thus, by monitoring the variables that lead to changes in blow flow and using those variables to control blood flow in real time can lead to better blood flow accuracy. This in turn can lead to higher average Kt/V for treatments with high blood flowrates.

In conclusion, preferred parameters being monitored by the processor to improve fluid flow rate measurement accuracy primarily include:
Fluid Flow Velocity (Pump Head Speed)
Inlet and Outlet Pressures
Tubing Aging

Additional parameters that may be monitored to improve the accuracy of determining the dialysate and blood flow rates include fluid temperatures, ambient temperature, atmospheric pressure, altitude from sea level, and tube elasticity. These parameters may be monitored, in other words measured, by sensors such as temperature sensors and/or inlet and outlet pressure sensors connected to the dialysate and/or blood flow path, which in turn are connected to the control processor. Alternatively, the parameters may be estimated such as by a clock within the processor that records a patient's treatment to estimate a tube's aging including its change in elasticity. Similarly, pump head speed, and an initial estimate of flow rate, can be provided directly by the pump motors through feedback to the processor.

Advantageously, the hemodialysis system is transportable, lightweight, easy to use, patient-friendly and capable of in-home use.

In addition, the hemodialysis system provides increased accuracy in determining the flow rate of the blood flow not previously provided by hemodialysis systems so as to provide enhanced patient safety.

Moreover, the increased accuracy in the blood flow rate can be used in a feedback loop to provide increased control to maintain, increase or decrease pump motor speed to maintain the blood flow rate at desired parameters.

Other features and advantages of the present invention will be appreciated by those skilled in the art upon reading the detailed description, which follows with reference to graphs, diagrams, and figures herein.

In closing, regarding the exemplary embodiments of the present invention as shown and described herein, it will be appreciated that a hemodialysis system is disclosed. The principles of the invention may be practiced in a number of configurations beyond those shown and described, so it is to be understood that the invention is not in any way limited by the exemplary embodiments, but is generally directed to a hemodialysis system and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention. Furthermore, the various features of each of the above-described embodiments may be combined in any logical manner and are intended to be included within the scope of the present invention.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the Specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present Specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

It should be understood that the logic code, programs, modules, processes, methods, and the order in which the respective elements of each method are performed are purely exemplary. Depending on the implementation, they may be performed in any order or in parallel, unless indicated otherwise in the present disclosure. Further, the logic code is not related, or limited to any particular programming language, and may comprise one or more modules that execute on one or more processors in a distributed, non-distributed, or multiprocessing environment.

While several particular forms of the invention have been illustrated and described, it will be apparent that various modifications can be made without departing from the spirit and scope of the invention. Therefore, it is not intended that the invention be limited except by the following claims. We claim:

1. A hemodialysis system comprising:
a machine housing;
an arterial blood line for connecting to a patient's artery for collecting blood from a patient;
a venous blood line for connecting to a patient's vein for returning blood to a patient;
a dialyzer;
a blood flow path connected to said arterial blood line and said venous blood line for transporting blood from a patient to said dialyzer and back to a patient;
a source of dialysate;
a dialysate flow path, isolated from the blood flow path, connected to said source of dialysate and said dialyzer for transporting dialysate from said source of dialysate to said dialyzer;
at least one dialysate pump for pumping dialysate through said dialysate flow path;
a blood pump for pumping blood through said blood flow path, said blood pump including motor;
one or more sensors for monitoring one or more operating parameters of the hemodialysis machine;
a control processor connected to said one or more sensors and said blood pump, said control processor possessing memory storing one or more algorithms which adjust the blood pump's motor speed to increase or decrease based upon receipt of said one or more operating parameters from said sensors so as to maintain a constant blood fluid flow through said blood flow path.

2. The hemodialysis system of claim 1 wherein:
said blood pump includes a rotatable motor;
said one or more sensors include a blood pump sensor which monitors the number of rotations of said motor;
said one or more operating parameters includes rotations of said motor and said one or more algorithms adjust the blood pump's motor speed to increase or decrease based upon the control processor's receipt of the number of rotations of said motor.

3. The hemodialysis system of claim 1 wherein:
said blood pump includes a rotatable motor which produces a pump head speed;
said one or more sensors include a blood pump sensor which monitors the pump head speed;
said one or more operating parameters include pump head speed and said one or more algorithms adjust the blood pump's motor speed to increase or decrease based upon the control processor's receipt of pump head speed.

4. The hemodialysis system of claim 1 wherein:
said one or more sensors include a blood inlet pressure sensor which monitors the blood inlet pressure; and
said one or more operating parameters include blood pump head speed and said one or more algorithms adjust the blood pump's motor speed to increase or decrease based upon the control processor's receipt of blood inlet pressure.

5. The hemodialysis system of claim 1 wherein:
said one or more sensors include a blood temperature sensor which monitors the blood's temperature; and
said one or more operating parameters include blood temperature and said one or more algorithms adjust the blood pump's motor speed to increase or decrease based upon the control processor's receipt of blood temperature.

6. The hemodialysis system of claim 1 wherein:
said one or more operating parameters are selected from the group consisting of blood pump rotations, blood pump head speed, and blood pump inlet pressure, and combinations thereof.

7. The hemodialysis system of claim 1 wherein:
said one or more operating parameters are selected from the group consisting of blood pump rotations, blood pump head speed, blood pump inlet pressure, blood temperature and combinations thereof.
